(19) United States
(12) Patent Application Publication
(10) Pub. No.: US 2021/0402399 A1
(43) Pub. Date: Dec. 30, 2021

(54) METHOD AND APPARATUS FOR REAL TIME MONITORING OF CELL AND TISSUE CULTURE

(71) Applicant: Stemnovate Limited, Cambridgeshire (GB)

(72) Inventors: Ruchi Sharma, Cambridgeshire (GB); Femi Akande, Cambridgeshire (GB)

(21) Appl. No.: 17/388,255

(22) Filed: Jul. 29, 2021

Related U.S. Application Data
(63) Continuation of application No. PCT/IB2020/050750, filed on Jan. 30, 2020.

(30) Foreign Application Priority Data
Jan. 30, 2019 (GB) 1901273.1

(51) Int. Cl. B01L 3/00; C12M 1/36

(52) U.S. Cl. CPC B01L 3/502715; C12M 41/48; B01L 2200/147; B01L 2400/0478; B01L 2200/0689; B01L 2200/0647; B01L 2300/0645

(57) ABSTRACT

The present invention provides apparatus for and a method of real time monitoring of cell or tissue based assays. The apparatus comprises a culture chamber. The culture chamber has at least an inlet port for introduction of fluids and an outlet port for removal of fluids, and a sealable enclosure for enclosing the culture chamber, wherein the culture chamber defines a micro-environment and the sealable enclosure defines a macro-environment around the micro-environment, and wherein each of the micro-environment and macro-environment is controllable independently of the other.

### CROSS REFERENCE TO RELATED APPLICATIONS

Continuation of International Application No. PCT/162020/050750 filed on Jan. 30, 2020. Priority is claimed from British Application No. 1901273.1 filed on Jan. 30, 2019. Both the foregoing applications are incorporated herein by reference in their entirety.

### FIELD OF THE INVENTION

This invention relates to a microfluidic device for real time monitoring of cell and tissue culture, in particular a device for monitoring cell and tissue cultures in cell based assays.

### BACKGROUND TO THE INVENTION

Microfluidic devices have the capability to use very small quantities (one billionth to one quadrillionth of a litre) of biological or chemical samples and reagents such as proteins, carbohydrates, DNA and cell or tissue samples. Prior art cell assay based microfluidic devices can analyse, separate and detect fluids contained within the cell or tissue based samples. This is achieved by manipulating fluids within a microfluidic network, which generally includes elements such as valves, gates, pumps, reaction chambers, mixing chambers, enrichment modules, filtration modules and detection modules. These elements can typically be thermally actuated under computer control.

Cell assay based microfluidic devices typically use a plastic tissue culture for studying drug effects, pharmacokinetics, pharmacodynamics and disease modelling. Such a configuration fails to accurately mimic cellular interaction which is essential for tissue specific functionality. Prior art cell assay based microfluidic devices thus are often not suitable for long term cell culture. It is partly for this reason, and the consequent lack of useful in vitro data, that most new drugs are tested on animals. While animal testing provides an indication of efficacy, toxicity and other parameters, the effect of a drug on animals cannot always be translated to humans.

Manufacturing constraints have resulted in the majority of microfluidic devices being formed from uniform, rectangular cross-sections. Despite the simplicity of most microfluidic devices, low quality devices are prevalent. Such low quality devices suffer from fluid flow disturbances caused by surface roughness of channels, poor response times and other inefficiencies resulting from oversized channels and chambers and biofouling.

Biofouling is a recognised limitation of microfluidic devices intended for use in cell culture. It is thus desirable to prevent bacteria, viruses and fungal spores from entering the microfluidic device.

In addition to biofouling, cell (or tissue) culture viability is negatively affected by variations in the environment of the cell assay. Variations in temperature in particular are problematic but are inherent in prior art microfluidic devices where long channels and large mixing chambers are used. Such a design leads to large thermal losses that are very difficult to control. Furthermore, sheer stresses on cells as the fluids pass through microfluidic devices can lead to damaged cells and failed cultures.

It is against this background that the invention has arisen.

### SUMMARY OF THE INVENTION

An aspect of the invention provides a microfluidic device for multi-parametric, real-time monitoring of an assay comprising: at least one culture chamber for performing an assay comprising: a chamber inlet port for the introduction of fluids; a chamber outlet port for the removal of fluids; and one or more electrodes configured to detect parameters within the culture chamber, the culture chamber defining a micro-environment; a sealable enclosure defining a macro-environment, for enclosing the at least one culture chamber, and one or more sensors for monitoring parameters of the macro-environment, wherein each of the micro-environment and macro-environment is controllable independently of the other.

Use of an independently controlled micro-environment and macro-environment aids in maintaining the environment of a cell or tissue based assay over extended periods of time. To ensure long term viability of a cell or tissue based assay it is essential that core parameters such as temperature, pressure, pH level and oxygen level, for example, are tightly controlled. Variations in one or more parameter could cause damage to or death of a cell or tissue sample. By providing a macro-environment around the micro-environment and permitting independent control of each, the micro-environment is less susceptible to variations in local environmental factors. Further, the independent control of both the micro-environment and macro-environment is beneficial in seeking to achieve homeostasis, i.e. only minor changes should be required to certain parameters of the micro-environment and macro-environment in order to maintain equilibrium of the condition and parameters of the cell or tissue sample.

In some embodiments, the sealable enclosure may comprise at least one environmental medium port for introducing and/or removing environmental medium from the sealed enclosure. These can be termed the enclosure inlet port and enclosure outlet port. The environmental medium may be air, gas, liquid, water or other suitable medium.

As described above, achieving homeostasis of both the micro-environment and macro-environment requires certain parameters to be varied in response to sensed changes. For example, if the temperature in the macro-environment starts to increase, a cooling medium may be introduced into the enclosure to prevent further rises in temperature and subsequent damage to or death of the cell culture. The provision of at least one environmental medium port allows for medium to be introduced to and/or removed from the enclosure. In some embodiments the enclosure, once sealed, is air tight or water tight.

In some embodiments, the device may further comprise one or more sensors for monitoring parameters of the micro-environment and/or cell or tissue sample. In particular, these sensors may take the form of electrodes. In some embodiments, the device may further comprise one or more sensors for monitoring parameters of the macro-environment. In some embodiments, the aforementioned sensors may include, but are not limited to, sensors configured to monitor: temperature, pressure, gas composition, pH level, electrochemistry and humidity.

In some embodiments, one or more sensors are located at or adjacent to the enclosure inlet port. In some embodiments, one or more sensors are located at or adjacent to the enclosure outlet port.

Positioning of sensors at or adjacent to the enclosure inlet port ensures that parameters of environmental medium can be monitored as the medium enters the enclosure. Consequently, the sensed parameters can be used to determine further required changes to the micro-environment and/or macro-environment. Positioning of sensors at or adjacent to the enclosure outlet port allows for the effect of changes to the micro-environment and/or macro-environment to be monitored in real time as environmental medium exits the sealed chamber and for further required changes to be addressed quickly.

In some embodiments, the device may further comprise a controller for making adjustments to the micro-environment and/or macro-environment in response to changes to parameters measured by the sensors, wherein the controller is programmed to maintain the micro-environment and macro-environment within the range of 0.1 to 5% of predetermined parameters, preferably within 3% of baseline parameters.

In some embodiments, the device may further comprise heating means located at or adjacent to the enclosure inlet port.

The heating means is used to heat environmental medium as it enters the sealed enclosure through the inlet port. It is important that the environmental medium is within a pre-defined temperature range. Therefore, the heating means located at or adjacent to the enclosure inlet port selectively heats the environmental medium as it enters the inlet port such that the environmental medium is at an optimal temperature as it enters the sealed enclosure. A valve associated with the inlet port may be configured to remain closed unless the environmental medium is determined to be within a pre-defined temperature range.

Rather than attempting to bond a cell or tissue based sample to a surface in-situ, embodiments of the invention permit the sample to prepared externally under controlled conditions on a pre-fabricated “chip”, referred to herein as the culture chamber. The pre-fabricated chip, once prepared, can be stored prior to insertion into the microfluidic device of the present invention. Such a configuration reduces the chances of premature cell or tissue death and allows researchers to prepare multiple samples at the same time.

In some embodiments, the device may further comprise a waste chamber downstream of the outlet port from the cell culture chamber.

The waste chamber stores waste environmental medium from the micro-environment and/or macro-environment or waste material associated with the cell or tissue sample. Monitoring of this waste allows for any effect on the micro-environment and/or macro-environment in response to a previous variation to be identified. Consequently, additional variations can be instructed and monitored in an attempt to achieve homeostasis, as described above.

As described herein, a substrate (such as a chip) containing a cell or tissue based assay may be used in the device or method of the invention. In some embodiments, the chip comprises a silicate material, such as glass. It will be understood that references herein to “chip” may also be referred to as “substrate”. In some embodiments, the surface of the chip is chemically modified. In a further embodiment, the surface of the chip is covalently bound to a polymer (e.g. a hydrogel). The chip may be modified by covalently binding polymers to the surface, such as by silanisation. Polymers bound to the surface of the chip may be further modified using a cross-linking agent.

In some embodiments, the polymer comprises a hydrogel. Suitable polymers for use in the hydrogel are known by those skilled in the art and can include, for example, poly(ethylene glycol), hyaluronic acid, gelatin, collagen, MATRIGEL®, dithiol polymers (e.g., acrylamide), poly(ethylene glycol)-diacrylate, poly(ethylene glycol)-vinyl sulfone, and the like. Particularly suitable polymers can be, for example, polyacrylamide.

In further embodiments, the hydrogel comprises a thin film, dome shape or flat top on the surface of the chip. It will be understood that the choice will depend on the nature of the experimental reagents to be tested or the cell morphology required. In some embodiments, the hydrogel comprises a diameter of at least 1 mm, such as about 1 to 12 mm. In some embodiments, the hydrogel comprises a height of at least 0.1 mm, such as about 0.1 to 2 mm.

Particularly suitable polymers can be, for example, functionalised polymers. Therefore, in some embodiments, the polymer comprises one or more functional groups. The functional group may be used to enable attachment to biomolecules of interest, such as DNA, RNA or proteins. Such biomolecules may also be important for interaction with the cell or tissue used in the cell or tissue based assay.

In some embodiments, the polymer is coated with one or more extracellular matrix (ECM) components, in particular one or more ECM proteins. ECM is the non-cellular component present within all tissues and organs. It provides physical scaffolding for the cellular constituents as well as containing essential biochemical molecules involved in tissue morphogenesis, differentiation and homeostasis. ECM consists of a broad spectrum of matrix proteins that vary depending on the cell type to be supported. Therefore, in some embodiments the ECM proteins comprise: fibronectin, vitronectin, laminin, collagen (types I-IV), elastin or a combination thereof. ECM components may also include proteoglycans, such as heparan sulphate, chondroitin sulphate and keratan sulphate, or other polysaccharides, such as hyaluronic acid. Applying one or more ECM factors and/or proteins to the chip enables simulation of the physiological stromal composition in various tissues.

In some embodiments, the polymer is coated with one or more physiologically active agents. Such agents include hormones, growth factors, enzymes, ligands and receptor growth factors. In particular, in some embodiments, the polymer is coated with one or more growth factors, for example, growth factors selected from: epidermal growth factor, insulin-like growth factor, transforming growth factor or platelet derived growth factor.

The modified chip comprising a polymer (hydrogel) may be used to encapsulate at least one cell. Therefore, in some embodiments, the chip comprises an organotypic cell culture of one or more cell types. In particular, the cell culture comprises mammalian cells, such as human or animal cells. In some embodiments, the cell culture is derived from a cell line. In some embodiments, the cell culture is derived from a stem cell, such as an induced pluripotent stem cell. As described herein, the surface of the chip may be modified to be covalently bound to a hydrogel which enhances organotypic cell culture thereby mimicking cellular and tissue physiology that is characteristic of complex tissue systems. References herein to “organotypic” refer to cells and/or tissues cultured under conditions in which they retain the cellular composition, morphology and the physiological properties of the organ from which they are derived.

The surface modification of the chip described herein can be used to simulate in vivo-physiology while also being manufactured cost effectively with high reproducibility to allow faster and efficient quantitative data acquisition. The chip can be used to support cell or tissue based assays for use in the invention, such as assays for studying drug effects (i.e. drug screening), mechanisms of toxicity (such as pharmacokinetics and pharmacodynamics) and disease modelling.

In some embodiments, the chip comprises a gasket, such as a silicon gasket. A gasket can be used to form a silanisation cell and to prevent leakage.

Another aspect of the invention provides a method of monitoring and controlling an assay in real-time, employing the microfluidic device according to the invention, comprising the steps:
a) generating the microenvironment by:
   (i) introducing the assay into the culture chamber; and
   (ii) inputting culture medium via the chamber input port and, where necessary, outputting waste culture medium via the culture output port;
b) placing the culture chamber into the enclosure to generate the macro-environment;
c) measuring at least one starting parameter obtained from the sensors to define a baseline;
d) monitoring the at least one starting parameter; and

if the at least one starting parameter deviates from a predetermined parameter by a value above or below a threshold, adjusting the micro-environment and/or the macro-environment accordingly, such that the at least one starting parameter returns to a value within the threshold.

Testing of new drugs on a cell or tissue sample requires the sample to be maintained under controlled, consistent conditions. It is therefore important to ensure that the conditions are optimal as soon as the sample is inserted into an assay. This aspect of the present invention thus requires that a micro-environment intended to house the sample is generated and that a first parameter of the micro-environment is controlled within a pre-determined range. Once the sample is inserted into the micro-environment, the first parameter is monitored and any variation in that parameter above or below a threshold is addressed by applying a correction step to the first parameter. Taking temperature as an example, assume that the optimal temperature in the micro-environment is 37 degrees Celsius. During the testing period the temperature starts to drop. The temperature is monitored and if the variation in temperature starts to approach say 3%, i.e. the temperature approaches 35.9 degrees Celsius a heater is activated to increase the temperature of the micro-environment.

A yet further aspect the invention provides a method of culturing cells comprising the steps:
a) introducing cells and culture medium to the culture chamber of the device according to the invention;
b) attaching the culture chamber to a syringe, a waste chamber and optionally an imaging device;
c) placing the culture chamber into the sealable enclosure;
d) connecting the device to a computing device configured to control the device, the syringe and the imaging device and monitor parameters obtained from the sensors and/or electrodes; and
e) maintaining parameters within predetermined thresholds.

Rather than making changes directly to the micro-environment, it is advantageous to house the micro-environment within a macro-environment and vary parameters of the macro-environment instead. Variations to the macro-environment result in gentler, staged variations of the micro-environment thus assisting in achieving homeostasis of the micro-environment. Such a configuration requires less frequent monitoring of the micro-environment as a minor change to the macro-environment, which is monitored continuously, would have little impact on monitored parameters of the micro-environment

In some embodiments, the method further comprises the steps of: measuring a third parameter associated with a cell or tissue based assay after correction of the first and/or second parameter; and further correcting the first and/or second parameter if the third parameter measurement deviates from a baseline by more than a threshold value.

It is advantageous to monitor the effect of a variation of the micro-environment and/or macro-environment on the cell or tissue sample. Waste medium associated with the sample is diverted to a waste well where one or more parameters are monitored. Any change in parameter is compared against a change that would have been expected to result from a variation to a parameter of the micro-environment and/or macro-environment.

### BRIEF DESCRIPTION OF THE FIGURES

For a better understanding of the invention and to show how the same may be carried into effect, there will now be described by way of example only, specific embodiments, methods and processes according to the present invention with reference to the accompanying drawings in which:

FIG. 1 shows a microfluidic device according to the invention.

FIGS. 2a-2c show an optimised schematics of the microfluidic device according to the invention, showing a fluid pathway modelled to optimise the flow of liquids into and out of the culture chamber in order to reduce sheer stress on cells within the chamber.

FIG. 3 shows an example layout of electrodes on the culture chamber according to the invention. The regions labelled a and b are various “detection zones” that can be customised according to cell type etc.

FIGS. 4a and 4b show ways in which the device may be multiplexed or run in parallel. In FIG. 4a there is a single point of fluid entry, flowing along 10 “spokes” to a total of 30 different culture chambers. FIG. 4b shows a more linear conformation in which each chamber might be supplied by its own syringe.

FIG. 5 shows a schematic of the computer controlled integrated system in which the device might be implemented.

FIG. 6a shows cell adherence and growth within the culture chamber with (A, B, C) and without (D, E, F) rock inhibitor. Flow through the chamber shows uniform adherence and distribution at 60 minutes post-seeding (4× magnification—A, D; 10× magnification—B, E and 20× magnification—C, F).

FIG. 6b shows cell attachment and distribution in the presence and absence of rock inhibitor on day 0, day 1 and day 2.

FIG. 7 shows the growth and differentiation of seed iPSC cells into hepatocyte-like cells grown in the device of the present invention, over time.

### DETAILED DESCRIPTION

Microfluidic devices generally include a substrate that defines one ore more microfluidic networks, each including one or more channels, process modules and actuators. Materials, e.g. samples and reagents, are manipulated within the microfluidic network(s), generally to determine the presence or absence of some target.

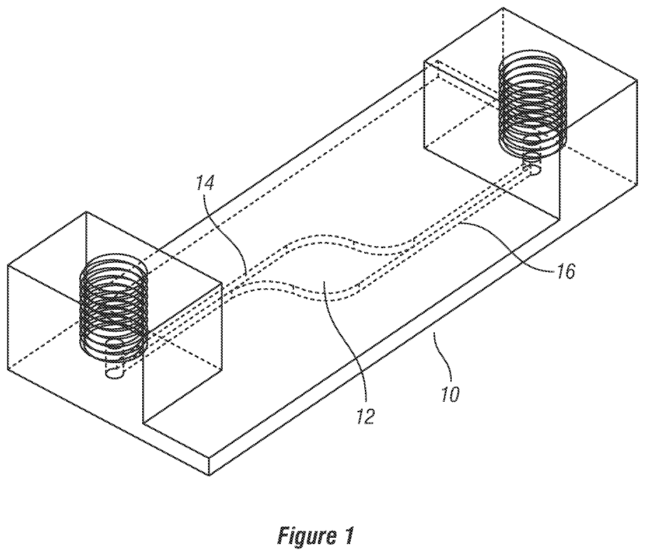

Referring to FIG. 1, there is shown a microfluidic device 8 used for cell or tissue culture. As shown in FIG. 1, the microfluidic device comprises a culture chamber 10, featuring a well 12 for receiving a cell or tissue sample. The sample is in fluid communication with a chamber inlet port 14 and a chamber outlet port 16. The microfluidic device 8 may also comprise one or more mixing chambers, valves and pumps for manipulating very small quantities of fluid within the microfluidic device 8 (not shown). Each of the inlet port 14 and outlet port 16 are shown aligned with the centre line of the microfluidic device 8 when viewed in plan. The culture chamber 10 comprises a well 12 which is positioned at the lateral and longitudinal intersection of the microfluidic device 8.

The culture chamber well 12 has a depth of approximately 600 microns and a diameter of approximately 7000 microns. The chamber 10, or just the well 12 may accommodate a hydrogel at the base upon which a cell or tissue sample is directly embedded and cultured. Hydrogels according to the present invention have a height of approximately 200 microns. The configuration of the chamber inlet 14 and chamber outlet 16 ensures that fluid entering and exiting the chamber 10 is uniformly distributed across the hydrogel to: a) ensure all cells embedded on the hydrogel receive adequate nutrients, b) ensure well distributed cells and c) to drain waste fluid from the chamber 10.

Long term viability of cell tissue samples requires that both a micro-environment and macro-environment are accurately controlled. A macro-environment is provided by enclosing the microfluidic device 8 within a sealable enclosure. The macro-environment is controlled through monitoring a plurality of parameters within the macro-environment by way of sensors including: temperature sensors, humidity sensors, CO₂ sensors and pressure sensors, for example. Additional sensors, which may be electrodes, that could be used in the micro-environment include sensors for monitoring oxygen levels, glucose concentrations, electrical impedance and pH within the sample, for example. Further parameters that may be monitored include growth factors, electrolytes and proteins.

The micro-environment refers to the internal environment of the microfluidic device.

Additionally, waste fluid medium from the cell culture chamber is monitored to determine the cell or tissue culture viability and any effect on the cell or tissue culture caused by a change to the micro-environment or macro-environment. A fluid conduit connects the outlet port of the cell culture chamber to a waste chamber 24. Certain parameters of the waste fluid are measured either by a sensor in or adjacent to the outlet port or by a sensor in or adjacent to the waste chamber.

The culture chamber 10 comprises electrodes 18 configured to measure changes in impedance or conductivity created by changes in the cell culture. The sensing region S can be subdivided for the purpose of monitoring individual cells, for example (as indicated by a and b).

The culture chambers can be multiplexed as shown in FIGS. 4a and 4b.

Each of the sensors referred to above is connected to a computing device 34 including processing means. The computing device 34 monitors variations to selected parameters and instructs changes to the micro-environment to maintain near consistent environmental parameters. For example, a heating element is provided within the micro-environment to ensure that the cell or tissue sample is kept at a constant temperature.

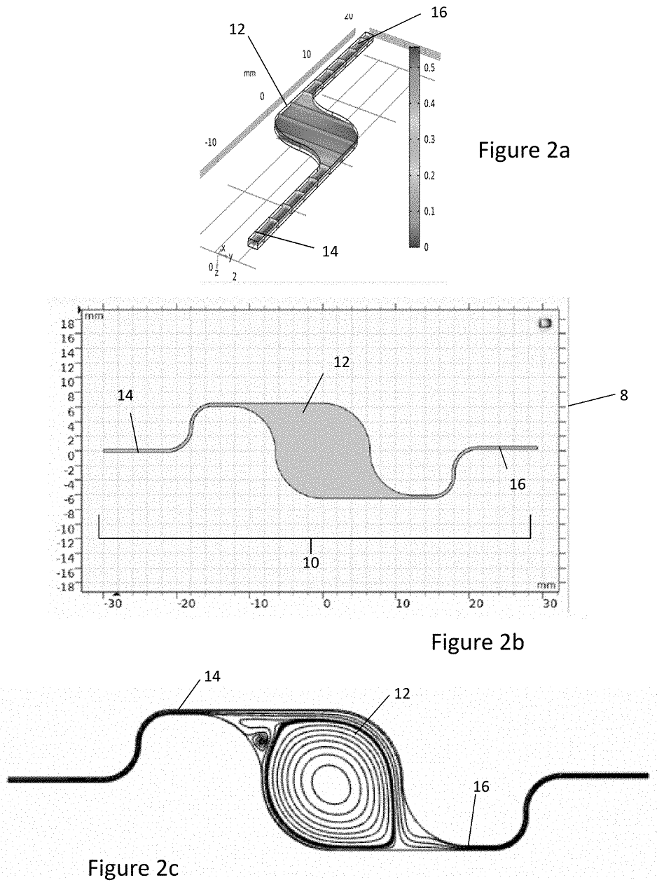

FIGS. 2a-2c show, respectively, optimised schematics of the microfluidic device according to the invention, showing a fluid pathway modelled to optimise the flow of liquids into and out of the culture chamber in order to reduce sheer stress on cells within the chamber.

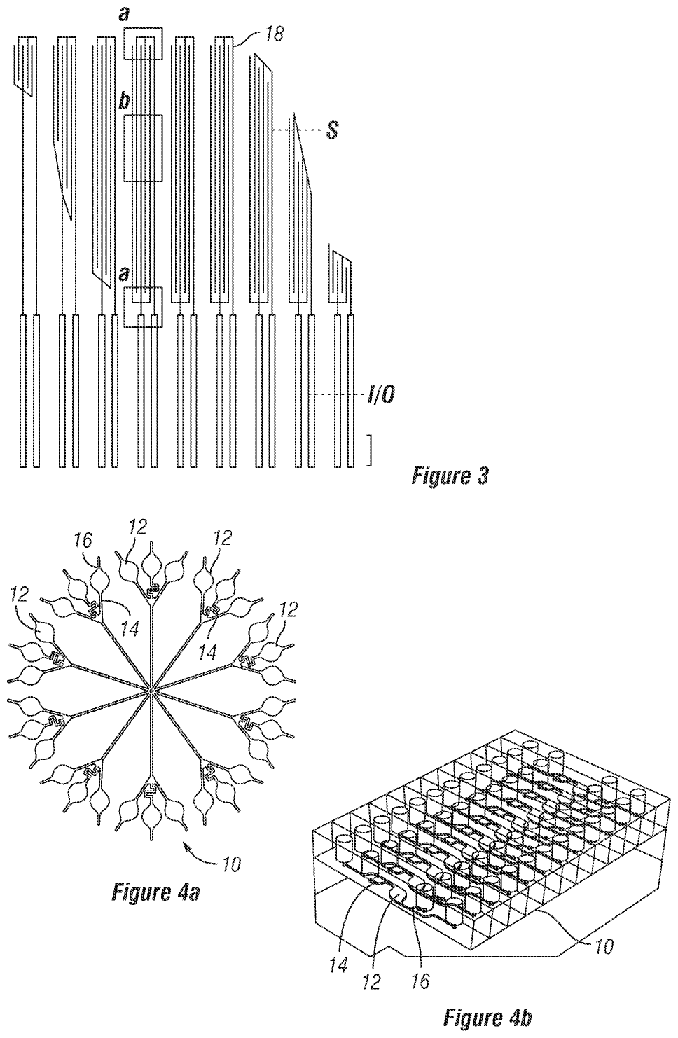

FIG. 3 shows an example layout of electrodes on the culture chamber according to the invention. The regions labelled a and b are various “detection zones” that can be customised according to cell type etc.

FIGS. 4a and 4b show ways in which the device may be multiplexed or run in parallel. In FIG. 4a there is a single point of fluid entry, flowing along 10 “spokes” to a total of 30 different culture chambers. FIG. 4b shows a more linear conformation in which each chamber might be supplied by its own syringe.

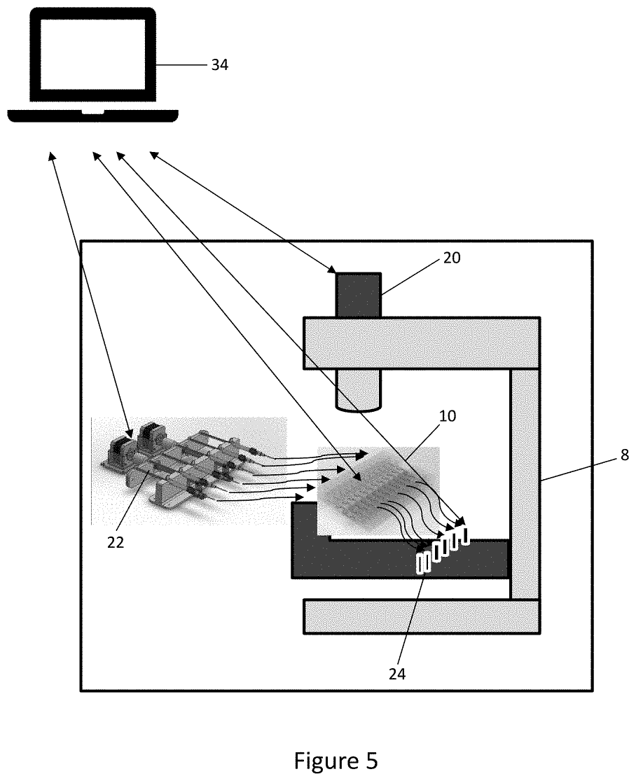

FIG. 5 shows a schematic of the computer controlled integrated system in which the device might be implemented.

The sealable enclosure comprises an enclosed jacketed chamber, i.e. an incubator. At least one holder for a microfluidic device 8 is provided within the sealed enclosure. The sealable enclosure may comprise multiple holders or a single holder configured to receive multiple microfluidic devices 10 (FIGS. 4b and 5). The sealable enclosure provides access ports for the inlet port and outlet port of the microfluidic device 8. The sealable enclosure also provides one or more perfusion holes for introduction or removal of environmental fluids, i.e. oxygen or heated medium. Each of the access ports and perfusion holes is closable by respective valves. A pump is also associated with each access port and perfusion hole. In some embodiments a pump can both introduce and remove fluid from the sealed enclosure or microfluidic device 8. In other embodiments pumps may either introduce or remove fluid from the sealed enclosure or microfluidic device 8.

In one embodiment, the sealable enclosure is at least partially transparent to provide visibility of the cell or tissue sample without needing to remove the sample from the enclosure. The sealable enclosure may also include one or more viewing areas for enabling inspection of the cell or tissue sample through a microscope. In other embodiments one or more cameras may be positioned within the sealable enclosure above the culture chamber 12. 20 is a microscope (imaging device) viewer, it can be viewed by a person or the image captured by computer and either analysed or pinged to a user via an app or something similar for remote viewing. 22 shows the various syringes for injecting fluid into the microfluidics chamber of the device.

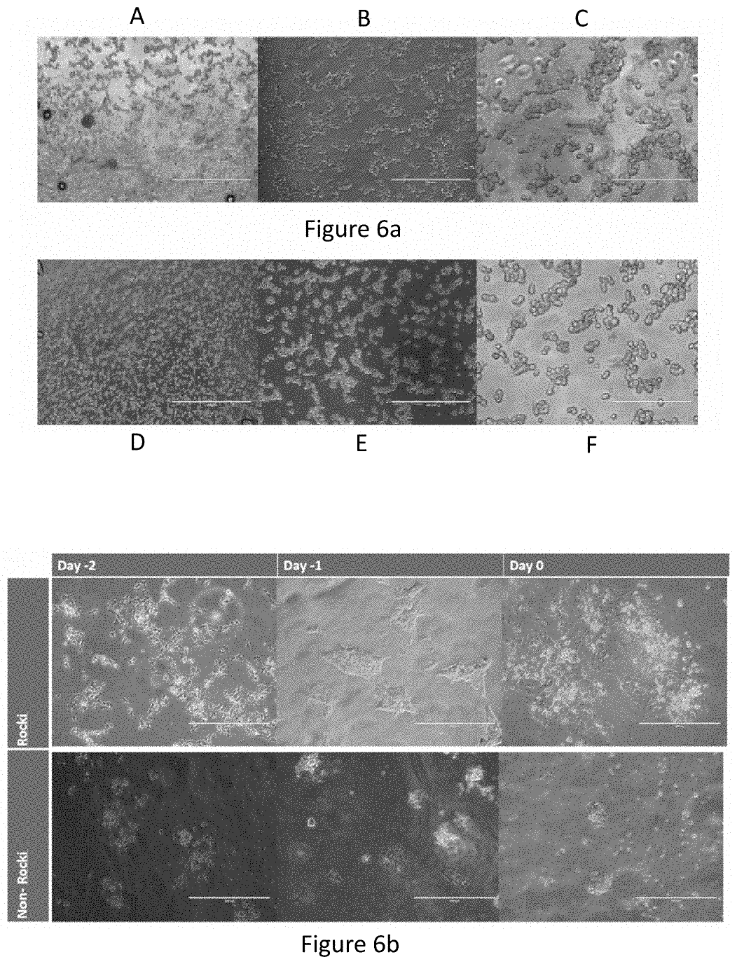

FIG. 6a shows cell adherence and growth within the culture chamber with (A, B, C) and without (D, E, F) rock inhibitor. Flow through the chamber shows uniform adherence and distribution at 60 minutes post-seeding (4× magnification—A, D; 10× magnification—B, E and 20× magnification—C, F).

FIG. 6b shows cell attachment and distribution in the presence and absence of rock inhibitor on day 0, day 1 and day 2.

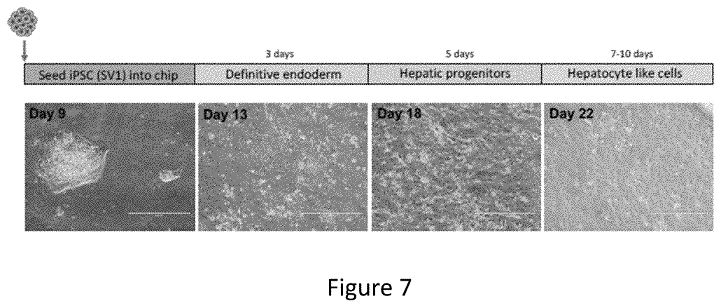

FIG. 7 shows the growth and differentiation of seed iPSC cells into hepatocyte-like cells grown in the device of the present invention, over time.

In general, the micro-environment and micro-environment are each prepared prior to introduction of a cell or tissue based assay. For example, the macro-environment may be prepared such that the temperature is 37 degrees Celsius, the humidity is 90%, the oxygen level is 21% and the CO₂ level is 5%. The micro-environment would be prepared dependent on the type of cell or tissue sample in the assay. It will be appreciated that the micro-environment and macro-environment can be modified as is necessary for a particular cell or tissue type and the values given are by way of example only.

Certain parameters of the micro-environment and macro-environment are measured to ensure that the measured values are within pre-defined threshold limits. This initial measurement is used to establish a baseline measurement for certain parameters of both the micro-environment and macro-environment. To prevent damage to or death of the cell or tissue sample the measured parameters of the micro-environment and macro-environment are monitored to identify any deviations from the baseline measurement for each parameter. The parameters of the micro-environment are generally measured every three to ten hours, preferably every four to six hours. However, depending on the nature of the sample and the experimental methodologies and protocols employed, the micro-environment may be monitored more or less often. For example, periods of cell culture that exceed one day may necessitate periodic monitoring every four to six hours whereas shorter periods of cell culture may necessitate more regular monitoring of every 1 to 15 minutes. The use of cameras additionally provides researchers with the ability to view cell cultures in real time without opening the sealable enclosure.

The parameters of the macro-environment are measured more often, preferably at least once per second. Sometimes, certain parameters of the macro-environment are monitored several times per second to provide real time observations of temperature and gas composition, for example. This data can be manipulated to provide average readings for the micro-environment as a whole. Localised parameters can be monitored through use of sensors located near the microfluidic device 8.

If a deviation from a parameter baseline measurement is determined the percentage deviation is calculated. Assuming that the deviation threshold is three percent above or below the parameter baseline measurement, a corrective action would be applied in respect of any measurement being identified as exceeding a deviation threshold value. In the case of the temperature of either the micro-environment or macro-environment deviating by more than three percent from the temperature baseline measurement, a heating means is instructed to either add heat energy to the relevant environment or to lower the amount of heat energy applied to the relevant environment for a pre-determined period of time.

Waste fluid medium from the culture chamber is measured for certain parameters periodically, i.e. every 15 minutes, to monitor the effect of a corrective action on the cell or tissue sample. If the effect on a parameter of the cell or tissue sample is determined to be outside of an acceptable value, a further corrective action is applied to the cell or tissue sample. The period between measurements depends on the length of the cell culture. For a 48 hour cell culture, a measurement period of once per hour may be sufficient. For longer cell cultures, a measurement period of once every 4 hours may suffice. The period between measurements of waste parameters is determined based on the nature and duration of the cell culture.

The purpose of monitoring parameters of the micro-environment, macro-environment and cell or tissue sample is to firstly keep parameters of both environments as constant as possible but also to monitor the cell or tissue sample and adjust the micro-environment and macro-environment as necessary to maintain cell or tissue viability.

Also described is a method of modifying the surface of a chip for organo-typic cell culture, in particular for use in the present invention. Said method comprises covalently binding a polymer, in particular a hydrogel, to the surface of the chip. Covalent binding of the polymer to the surface of the chip may comprise silanisation, in particular when the chip comprises a surface containing hydroxyl groups, such as a silicate material, for example glass. Silanisation comprises functionalizing the surface of the chip with a silane solution. This results in the formation of a silane monolayer which acts as a coupling agent between the polymer and surface of the chip.

The surface of the chip may be first treated, such as by UV or ozone plasma treatment, to generate activated hydroxyl groups. Successful treatment of the chip surface (i.e. to provide activated hydroxyl groups) can result in improved wetting, low contact angle, improved adhesiveness and/or display of hydrophilic property. Therefore, modified surfaces may be confirmed by a hydrophilicity test and/or assessment of contact angle upon wetting.

Once treated, the surface of the chip is incubated with a silane solution. A suitable silane solution can be prepared with an alkoxysilane that is dissolved in an anhydrous organic solvent such as, for example, toluene. Suitable alkoxysilanes can be for example, aminosilanes, glycidoxysilanes and mercaptosilanes. Particularly suitable aminosilanes can be, for example, (3-aminopropyl)-triethoxysilane, (3-aminopropyl)-diethoxy-methylsilane, (3-aminopropyl)-dimethyl-ethoxysilane and (3-aminopropyl)-trimethoxysilane. Particularly suitable glycidoxysilanes can be, for example, (3-glycidoxypropyl)-dimethyl-ethoxysilane. Particularly suitable mercaptosilanes can be, for example, (3-mercaptopropyl)-trimethoxysilane and (3-mercaptopropyl)-methyl-dimethoxysilane. In one embodiment, the silane solution comprises 3-(trimethoxylsilyl) propyl methacrylate. The method may be performed at room temperature.

In some embodiments, the method comprises silanising the surface of the chip and then incubating the chip with the polymer (e.g. hydrogel). Incubation may comprise applying the polymer to the surface of the chip for at least 1 hour at room temperature. Polymers for use as hydrogels for application to the surface of the chip are commercially available or may be made using methods known in the art, for example dissolving a polymer in solution before applying a cross-linker and stabiliser for polymerisation.

In some embodiments, the method comprises sterilising the surface of the chip (i.e. to remove micro-contaminants or debris) prior to application of the polymer. Sterilisation may comprise UV-irradiating the surface of the chip.

In some embodiments, the method additionally comprises applying extracellular matrix components and/or proteins to the polymer on the surface of the chip.

The method may further comprise contacting the polymer (i.e. hydrogel) on the surface of the chip with a cell. This embodiment refers to seeding the cells with the purpose of culturing the cells. As known by those skilled in the art, a cell suspension is typically transferred to a surface of the chip and cells are given sufficient time to adhere. The chip comprising the covalently bound polymer and cell may then be transferred to the microfluidic device described herein.

In some embodiments, the method additionally comprises removing the polymer from the surface of the chip after use in the method/device described herein. This embodiment allows assessment of the cell population and/or cell environment for subsequent downstream molecular analysis, such as in gene expressions studies.

As employed herein microfluidic refers to a device for the manipulation and precise control of fluids that are geometrically constrained to a small volume. Typically, the volumes involved are sub-millilitre, that is, 1 ml and less.

Multi-parametric as employed herein refers to the measuring of multiple parameters. Typically, this is contemporaneous or simultaneous measuring of different parameters. The measurement may be continuous or intermittent. Typical parameters include conductance, impedance, pH, metabolic activity, turbidity, dissolved gases, among others.

Real-time as employed herein refers to the measurement of parameters at the time of, or shortly following, their occurrence.

Assay as employed herein refers to an investigative procedure to determine the quality and/or quantity of an analyte. In a broader context, assay can be analogous to experiment.

Culture chamber as employed herein refers to a millilitre or sub-millilitre size chamber in the device, through which fluids can be fed. Typically, fluids enter the culture chamber via an inlet port and exit via an outlet port. Generally, an assay will be run within the culture chamber.

In one embodiment the culture chamber has a volume of 1 ml or less.

In one embodiment the culture chamber has a volume in the range 0.1 μm to 1 ml.

Typically, fresh culture medium is introduced to the culture chamber via the inlet port and waste medium exits the culture chamber via the outlet port. It will be appreciated that the waste medium may provide valuable information about the assay occurring within the culture chamber and therefore it may be desirable to capture the waste medium for further analysis.

Electrodes as employed herein refer to one or more electrodes present in the culture chamber. The electrode(s) non-invasively monitor parameters within the chamber. For example, where the culture chamber contains cells, the electrode may monitor the cells and their physiological changes in vitro. Accurately measuring impedance is a particularly beneficial parameter that can be measure by the electrodes.

In some embodiments the electrodes are laid down on a substrate within the culture chamber by coating the substrate with Cr and Au in an optimised, predetermined pattern. The electrode(s) is wire bonded or conductive tapes are used for electrical conductivity measurement.

In some embodiments the gap between electrodes is in the range 10 micrometres to 100 micrometres.

In some embodiments the pitch of the electrodes is in the range 0.1 mm to 1 mm.

Micro-environment as employed herein refers to the small, relatively contained environment within the culture chamber. In contract, macro-environment as employed herein refers to a larger environment surrounding the micro-environment. Typically, the macro-environment is also contained as it is desired to control the macro-environment as a means of creating a “buffer” around the micro-environment.

In some embodiments the device comprises one or more sensors for monitoring parameters of the macro-environment.

It is useful to monitor the macro-environment in addition to the micro-environment because this information can be used to provide a finer degree of control over the micro-environment.

Advantageously, by providing a macro-environment around the micro-environment the micro-environment can be insulated from sudden changes in, for example, temperature.

In one embodiment the device further comprises a controller for making adjustments to the micro-environment and/or macro-environment in response to changes to parameters measured by the sensors or electrodes, wherein the controller is programmed to maintain the micro-environment and/or macro-environment within the range of 0.1 to 5% of predetermined parameters.

In some embodiments the adjustments are made in response to parameters detected in waste culture medium.

Waste culture medium as employed herein refers to culture medium or fluid that is exiting the culture chamber via the outlet port. It is beneficial to monitor the waste culture medium for metabolites, toxins and other indicators of what is happening inside the chamber. For example, monitoring for toxins emitted in the waste medium may be an indicator of the health of cells being cultured in the chamber. Likewise, pH, dissolved gases, turbidity and many other parameters may be monitored.

In one embodiment the sensors are selected from the group consisting: temperature, pressure, pH, glucose, gas composition, and humidity sensors.

In one embodiment one or more of the sensors are located at or adjacent to the enclosure inlet port.

In one embodiment one or more of the sensors are located at or adjacent to the enclosure outlet port.

Generally, the macro-environment is equipped with inlet and outlet ports, through which, for example, water or air can be added/removed. For example, warm water may be added to create a water bath around the culture chamber micro-environment to gently warm it. By doing this, rather than, for example, warming the culture medium directly, changes within the micro-environment can be more steadily adjusted and maintained.

In one embodiment the device comprises heating means located at or adjacent to the enclosure inlet port.

In one embodiment the device comprises a waste chamber downstream of the chamber outlet port.

The device disclosed herein may form part of a larger system which may be provided as an integrated system. Thus, in some embodiments the device further comprises a syringe in fluid communication with the chamber inlet port, for precise introduction of fluid to the culture chamber.

The syringe may be manually or automatically actuated and may be computer controlled, thereby providing remote control (for example, from a location away from the device).

Additionally or alternatively, the device may further comprise an imaging device, such as a microscope, integrated inside the system.

Thus in one embodiment the device further comprises an imaging device configures to image the culture chamber.

Advantageously, imaging the culture chamber permits real-time monitoring of the assay without the need to remove the culture chamber/micro-environment from the macro-environment. Similarly to the syringe, the imaging device may be manually or automatically adjusted and may be computer controlled, thereby providing remote control.

In some embodiment the imaging device is movable in XYZ directions and may be motorised to control these movements. Generally, the imaging device moves while the device (culture chamber) remains fixed in position.

In some embodiments, the system described may comprise multiple devices or multiple culture chambers within the enclosure. Advantageously, the imaging device is moveable between the individual culture chambers. In general, each culture chamber would be connected to its own syringe and have its own waste chamber.

In some embodiments the device comprises a plurality of culture chambers running in parallel, each defining a micro-environment independent of the others.

As employed herein, in parallel may mean physically parallel or, more likely that the assays are run simultaneously or contemporaneously.

The assay housed inside the culture chamber may be a two dimensional assay or a three dimensional assay.

For two dimensional assays, the surface of the culture chamber in modified using methods known in the art. For example, surface modification of borosilicate glass, ceramics, plastics (e.g. polycarbonate, polystyrene, PMMA) and a combination of extracellular matrix to allow cell, tissue or organ-specific culture. In some examples, the assay is not cell based, for example, it may be applicable for the synthesis of nucleotides.

For three dimensional assays, biological matrix protein gels or synthetic hydrogels in the culture chamber host cells or tissues. Additionally, the gel may host multiple cell types in defined regions. For example, liver cell differentiation is suited to this design. Design features of the culture chamber allow cell adherence, uniform distribution and directed differentiation via the selective addition of chemically defined media with growth factors and small molecules. The three dimensional assay can be implemented to host organoids.

The shape of the culture chamber may be adjusted to achieve different flow patterns of fluids from the inlet port to the outlet port. Different chamber shapes can lead to significantly different flow patterns and sheer stresses on the culture chamber. Certain shapes have been found to be particularly beneficial for assays requiring cell seeding and distribution.

The device described herein in particularly useful for the use in prolonged cell culture, for example, over several hours or days.

The device of the present invention has been shown to provide good cell distribution within the culture chamber and to enable cell differentiation (see FIGS. 5 and 6). Advantageously, this permits “organ-like” cell culture in which differentiated cells develop to provide a more “in vivo-like” environment. The device has been shown to provide differentiated liver cells, such as hepatocytes, kupffer cells etc. This organ-like (or organoid) system permits cells culture to the challenged by introducing drug candidates, for example, which can be screened for liver toxicity.

Further advantageously, the device and the optional system around the device (e.g. the syringe, imaging device etc.) permits remote control of the device. For example, via remote login to the computer system controlling the automation. Alternatively, software can be employed to monitor parameters and automatically correct them if a threshold is breached. Or, to raise an alert if an error occurs. The presence of an imaging device, that may be remotely accessed, permits viewing from anywhere in the world, for example.

It will be appreciated by those skilled in the art that although the invention has been described by way of example with reference to several embodiments, it is not limited to the disclosed embodiments and that alternative embodiments could be constructed without departing from the scope of the invention as defined in the appended claims.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

1. A microfluidic device for multi-parametric, real-time monitoring of an assay comprising:
at least one culture chamber for performing an assay comprising:
a chamber inlet port for the introduction of fluids;
a chamber outlet port for the removal of fluids; and
one or more electrodes configured to detect parameters within the culture chamber,
the culture chamber defining a micro-environment;
a sealable enclosure defining a macro-environment, for enclosing the at least one culture chamber, and
one or more sensors for monitoring parameters of the macro-environment,
wherein each of the micro-environment and macro-environment is controllable independently of the other.

2. A microfluidic device according to claim 1 further comprising an enclosure inlet port and an enclosure outlet port.

3. A microfluidic device according to claim 1 further comprising a controller for making adjustments to the micro-environment and/or macro-environment in response to changes to parameters measured by the sensors, wherein the controller is programmed to maintain the micro-environment and/or macro-environment within the range of 0.1 to 5% of predetermined parameters.

4. A microfluidic device according to claim 1 wherein the sensors are selected from the group consisting: temperature, pressure, glucose, gas composition, and humidity sensors.

5. A microfluidic device according to claim 2 wherein one or more of the sensors are located at or adjacent to the enclosure inlet port.

6. A microfluidic device according to claim 2 wherein one or more of the sensors are located at or adjacent to the enclosure outlet port.

7. A microfluidic device according to claim 2, further comprising heating means located at or adjacent to the enclosure inlet port.

8. A microfluidic device according to claim 1 further comprising a waste chamber downstream of the culture chamber.

9. A microfluidic device according claim 1 wherein the culture chamber has a volume of 1 ml or less.

10. A microfluidic device according to claim 1 further comprising a syringe in fluid communication with the chamber inlet port, for precise introduction of fluid into the culture chamber.

11. A microfluidic device according claim 1 further comprising an imaging device configured to image the culture chamber.

12. A microfluidic device according to claim 1 wherein the device comprises a plurality of culture chambers, each defining a micro-environment independent of the others.

13. A microfluidic device according to claim 1 wherein the one or more electrodes detect changes in conductance or impedance in the culture chamber.

14. A microfluidic device according to claim 1, wherein the surface of the culture chamber has been modified to comprise a covalently bound polymer.

15. A microfluidic device according to claim 1 further comprising an assay.

16. A method of monitoring and controlling an assay in real-time, employing the microfluidic device according claim 1, the method comprising:
a) generating the micro-environment by:
(iii) introducing the assay into the culture chamber; and
(iv) inputting culture medium via the chamber input port and, where necessary, outputting waste culture medium via the culture output port;
b) placing the culture chamber into the enclosure to generate the macro-environment;
c) measuring at least one starting parameter obtained from the sensors to define a baseline;
d) monitoring the at least one starting parameter; and
e) if the at least one starting parameter deviates from a predetermined parameter by a value above or below a threshold, adjusting the micro-environment and/or the macro-environment accordingly, such that the at least one starting parameter returns to a value within the threshold.

17. A method of culturing cells comprising:
a) introducing cells and culture medium to the culture chamber of the device according to claim 1;
b) attaching the culture chamber to a syringe, a waste chamber and optionally an imaging device;
c) placing the culture chamber into the sealable enclosure;
d) connecting the device to a computing device configured to control the device, the syringe and the imaging device and monitor parameters obtained from the sensors and/or electrodes; and
e) maintaining parameters within predetermined thresholds.
